(19) United States
(12) Patent Application Publication
(10) Pub. No.: US 2026/0041540 A1
(43) Pub. Date: Feb. 12, 2026

(54) METHOD OF MANUFACTURING TREATMENT APPARATUS FOR LIFTING SKIN AND TREATMENT APPARATUS FOR LIFTING SKIN MANUFACTURED USING SAME

(71) Applicant: META PHARM CO., LTD., Seoul (KR)

(72) Inventors: Su Jin LEE, Seoul (KR); Young Jik LEE, Hwaseong-si (KR); Bo Sung KANG, Gumi-si (KR); Jun Seob BYON, Gwangju-si (KR); Soo Man PARK, Seoul (KR); Kyu Hwa JUNG, Seoul (KR); Sung Jun LEE, Seoul (KR); Han Su YOO, Busan (KR); Hee Geon YOU, Suwon-si (KR); Jae Bong KIM, Seoul (KR)

(73) Assignee: META PHARM CO., LTD., Seoul (KR)

(21) Appl. No.: 19/363,606

(22) Filed: Oct. 20, 2025

Related U.S. Application Data
(63) Continuation of application No. PCT/KR2025/004949, filed on Apr. 11, 2025.

(30) Foreign Application Priority Data
Apr. 12, 2024 (KR) 10-2024-0049368

(51) Int. Cl. A61F 2/00; A61B 17/00; A61B 17/06

(52) U.S. Cl. CPC A61F 2/0059; A61B 17/06166; A61B 2017/00526; A61B 2017/00792; A61B 2017/06176

(57) ABSTRACT

A method of manufacturing a treatment apparatus for lifting skin according to the present disclosure and a treatment apparatus for lifting skin manufactured using the same allow a thread for a skin lifting treatment, having a plurality of barbs, to be formed within a cannula for commercial sale SO that separate manufacturing and installation of the thread into the cannula are unnecessary. Therefore, the thread may be sold with the cannula in a configuration formed within the cannula, thereby simplifying the manufacturing process.

### TECHNICAL FIELD

The present disclosure relates to a method of manufacturing a treatment apparatus for lifting skin and a treatment apparatus for lifting skin manufactured using the same, and more particularly, to a method of manufacturing a treatment apparatus for lifting skin in which a thread is inserted into the skin via a cannula for the purpose of lifting the skin, and a treatment apparatus for lifting skin manufactured using the same.

### BACKGROUND ART

In general, as the human body ages, skin aging progresses due to the loss of collagen, a component of the skin that regulates skin elasticity. As skin aging progresses, gravity causes the skin to gradually sag.

Methods for skin lifting treatment include laser lifting, which uses lasers, and thread lifting, which uses threads. Among these methods, thread lifting is a method for skin lifting treatment in which a biocompatible, absorbable thread is directly inserted into the skin and pulled to lift sagging skin. This method also regenerates collagen within the skin to restore skin elasticity and provides long-lasting skin lifting effects. Because thread lifting does not require skin incisions, thread lifting does not leave large scars on the skin and allows relatively immediate return to daily activities. For these reasons, thread lifting has recently become a widely used lifting method.

The thread used for the thread lifting includes a smooth thread and a thread having a plurality of cogs formed on the outer circumferential surface. When the thread having the plurality of cogs formed on the outer circumferential surface is inserted into the skin for lifting, the plurality of cogs engage the skin and pull the skin in the lifting direction. Accordingly, this thread may provide an enhanced skin lifting effect compared to the smooth thread.

Korean Patent No. 10-1057377 (published on August 17, 2011) (hereinafter referred to as the “related art”) discloses a “Medical Suture with Micro Cogs on the Surface and producing method for the same.” The related art includes 1) manufacturing a suture preform where micro cogs are formed on a surface thereof by heating and pressing a raw material of a suture for surgery in an overflow mould at a temperature condition from a melting point of the raw material of the suture to a glass transition temperature at a pressure condition from 10 kgf/cm2 to 200 kgf/cm2 in a heat-press solid-phase forming method and 2) manufacturing a suture with twist maintained by heating the suture preform and applying a tensile force and a rotational force to the suture preform in a vacuum state where the temperature condition from the melting point of the raw material of the suture to the glass transition temperature is maintained.

In general, when both ends of the polymer suture are fixed to prevent shrinkage or deformation even during a heating phase and the same range of heat is applied, the polymer suture exhibits increased ductility and maintains a tensile strength equal to that of the original thread of the polymer suture.

The related art utilizes the above-described characteristics of the polymer suture. In this method, the original thread is heated to a specific temperature below the melting point of the raw material of the polymer suture to increase ductility. When both ends of the polymer suture are fixed to prevent shrinkage or deformation and compression molding is performed to form protrusions on the surface, micro cogs may be formed on the surface of the original thread while maintaining the molecular orientation of the polymer suture.

However, the related art discloses a technique for manufacturing a polymer suture having the micro cogs formed thereon. The final product may be the polymer suture sold alone. However, for the physician's convenience during treatment, the polymer suture is also sold coupled to a cannula.

That is, in the related art, for final product sale, the manufactured polymer suture is inserted through and coupled to the cannula. The polymer suture, coupled to the cannula, is placed in a sterilization chamber and heated to a predetermined temperature for sterilization. The polymer suture is sold in a state coupled to the cannula.

Therefore, in the related art, the manufacturing process of a treatment apparatus for lifting skin is complex because the process of manufacturing a polymer suture having the micro cogs formed thereon is separate from the process of Coupling the polymer suture to the cannula for sterilization.

### DISCLOSURE

### Technical Problem

A technical object of the present disclosure is to provide a method of manufacturing a treatment apparatus for lifting skin and a treatment apparatus for lifting skin manufactured using the same, in which a thread for a skin lifting treatment, having a plurality of barbs, is formed within a cannula for commercial sale. Since the thread is formed within the cannula, there is no need to manufacture the thread and the cannula separately and install the thread in the cannula. Therefore, the thread may be sold with the cannula in a configuration formed within the cannula, thereby simplifying the manufacturing process.

The technical objects of the present disclosure are not limited to the aforementioned technical object, and other technical objects, which are not mentioned above, may be clearly understood by those skilled in the art from the following descriptions.

### Technical Solution

In order to achieve the above-mentioned object a method of manufacturing a treatment apparatus for lifting skin according to the present disclosure includes steps S1, S2, S3, S4, S5, and S6. In step S1, a preform of a thread for a skin lifting treatment may be inserted in a front-to-rear direction through a cannula having a handle at a rear end portion. In step S1, the preform of the thread may have a plurality of barb preforms protrudingly formed on the outer circumferential surface. In step S2, a front end portion of the preform of the thread protruding from a front end portion of the cannula may be fixed to the front end portion of the cannula using a first holder. In step S3, the preform of the thread may be twisted by rotating a rear end portion of the preform of the thread protruding from the handle. In step S4, the rear end portion of the preform of the thread may be pulled to tension the preform of the thread and fixed to the handle using a second holder. In step S5, the cannula may be covered with a cap to couple the rear end portion of the cap to the handle. In step S6, a predetermined temperature may be applied to the cannula to maintain a twist of the preform of the thread and to form the plurality of barb preforms into the plurality of barbs.

The predetermined temperature may be a sterilization temperature of the thread.

The sterilization temperature may range from 38° C. to 60° C.

In step S6, sterilization time may range from 90 minutes to 180 minutes.

In step S6, the cannula, covered with the cap and contained in sterilization packaging, may be placed in a sterilization chamber, and the predetermined temperature may be applied.

The plurality of formed barbs in step S6 may include a lifting surface configured to engage a user's internal skin tissue between the barbs and the thread body. The lifting surface may include a concave first bent surface.

A treatment apparatus for lifting skin according to the present disclosure is manufactured using the method of manufacturing a treatment apparatus for lifting skin.

The treatment apparatus for lifting skin according to the present disclosure includes a cannula, a thread for a skin lifting treatment, a first holder, and a second holder. The cannula may have a handle at a rear end portion. The thread may be inserted in a front-to-rear direction through the cannula. The thread may have a plurality of barbs protrudingly formed on an outer circumferential surface. The first holder may be configured to fix a front end portion of the thread protruding from a front end portion of the cannula to the front end portion of the cannula. The second holder may be configured to fix a rear end portion of the tread protruding from the handle to the handle. The thread may be formed such that the portion between the first holder and the second holder is formed as twisted thread, and the front end portion relative to the first holder and the rear end portion relative to the second holder are formed as untwisted thread.

The plurality of barbs may include a lifting surface, an inclined surface, a parallel surface, and both side surfaces. The inclined surface may be arranged on a side opposite the lifting surface and inclined relative to a length of the thread body. The parallel surface may connect the terminal edges of the lifting surface and the inclined surface. The parallel surface may be parallel to the length of the thread body. The both side surfaces may have planar configurations.

A concave first bent surface may be formed on the lifting surface of the plurality of barbs.

The lifting surface may include a first lifting surface and a second lifting surface. The first lifting surface may be arranged closer to the thread body relative to the first bent surface. The second lifting surface may be arranged farther from the thread body relative to the first bent surface. The first lifting surface and the second lifting surface may be arranged and inclined at different angles relative to the length of the thread body.

An interior angle of the first lifting surface relative to the length of the thread body may be at least twice an interior angle of the second lifting surface relative to the length of the thread body.

A protrusion length of the second lifting surface may be longer than a protrusion length of the first lifting surface.

Respective lateral portions of the lifting surface, the inclined surface, and the parallel surface may have a convex rounded shape.

The inclined surface may include a concave second bent surface.

The inclined surface may include a first inclined surface and a second inclined surface. The first inclined surface may be arranged closer to the thread body relative to the second bent surface. The second inclined surface may be arranged farther from the thread body relative to the second bent surface. The first inclined surface and the second inclined surface may be arranged and inclined at different angles relative to the length of the thread body.

An interior angle of the first inclined surface relative to the length of the thread body may be greater than an interior angle of the second inclined surface relative to the length of the thread body.

A protrusion length of the second inclined surface may be longer than a protrusion length of the first inclined surface.

The details of other embodiments are included in the detailed description and drawings.

### Advantageous Effects

With the method of manufacturing a treatment apparatus for lifting skin according to the present disclosure and the treatment apparatus for lifting skin manufactured using the same, a thread for a skin lifting treatment, having a plurality of barbs, to be formed within a cannula for commercial sale so that separate manufacturing and installation of the thread into the cannula are unnecessary. Therefore, the thread may be sold with the cannula in a configuration formed within the cannula, thereby simplifying the manufacturing process.

Effects of the present disclosure are not limited to the aforementioned effects, and the other effects not described above may be evidently understood by those skilled in the art from the claims.

### BRIEF DESCRIPTION OF DRAWINGS

FIG. 1 is a perspective view of a treatment apparatus for lifting skin according to an embodiment of the present disclosure.

FIG. 2 is an exploded perspective view of FIG. 1.

FIG. 3 is a block diagram illustrating a process according to a method of manufacturing a treatment apparatus for lifting skin according to an embodiment of the present disclosure.

FIG. 4 is a view illustrating a process according to a method of manufacturing a treatment apparatus for lifting skin according to an embodiment of the present disclosure.

FIG. 5 is a view illustrating a state in which a thread for a skin lifting treatment of a treatment apparatus for lifting skin according to an embodiment of the present disclosure is inserted into a piece of meat.

FIG. 6 is a graph illustrating the resistance force measured when a thread for a skin lifting treatment having a plurality of conventional 2D-shaped barbs is inserted in the same manner as in FIG. 5, with the meat piece fixed to a clamp of a tensile tester and the thread pulled in the direction of the arrow.

FIG. 7 is a graph illustrating the resistance force measured when a thread for a skin lifting treatment having a plurality of 3D-shaped barbs according to the present disclosure is inserted in the same manner as in FIG. 5, with the meat piece fixed to a clamp of a tensile tester and the thread pulled in the direction of the arrow.

FIG. 8 is a perspective view of the thread for a skin lifting treatment illustrated in FIGS. 1 and 2.

FIG. 9 is an enlarged view of a portion of a first, second, and third regions illustrated in FIG. 8.

FIG. 10 is an enlarged view of a portion of the first region illustrated in FIG. 8.

FIG. 11 is an enlarged view of a portion of the second region illustrated in FIG. 8.

FIG. 12 is a left-side view of FIG. 10, illustrating a first barb formed in the first region.

FIG. 13 is a left-side view of FIG. 11, illustrating a second barb formed in the second region.

FIG. 14 is a perspective view of a 1-2 barb illustrated in FIGS. 10 and 11.

FIG. 15 is a rear-lower perspective view of the 1-2 barb illustrated in FIGS. 10 and 11.

FIG. 16 is an enlarged view of the 1-2 barb illustrated in FIGS. 10 and 11.

FIG. 17 is a schematic view of the 1-2 barb illustrated in FIG. 16.

### DESCRIPTION OF REFERENCE NUMERALS

1: Thread for a skin lift treatment
2: Preform of thread for a skin lift treatment
6: Cannula
6A: Handle
7: First holder
8: Second holder
9: Cap
200, 300: Barb
230: Lifting surface
233: First bent surface
1000: Treatment apparatus for lifting skin

### MODE FOR DISCLOSURE

Hereinafter, A thread for a skin lifting treatment according to an embodiment of the present disclosure will be described with reference to the drawings.

It should be noted that when assigning reference numerals to components in each drawing, the same numerals are used for the same components as much as possible, even if the components are shown in different drawings. In addition, in describing embodiments of the present disclosure, detailed descriptions of related known configurations or functions are omitted if it is determined that such detailed descriptions would hinder understanding of the embodiments of the present disclosure.

The terms such as first, second, A, B, (a), and (b) may be used to describe components of the embodiments of the present disclosure. Such terms are used merely to distinguish the components from other components, and the nature, order, or sequence of the corresponding components is not limited by these terms. In addition, unless otherwise defined, all terms used herein, including technical and scientific terms, have the same meanings as commonly understood by those skilled in the art to which the present disclosure pertains. Terms such as those defined in generally used dictionaries should be interpreted as having meanings consistent with the contextual meanings in the related technology, and should not be interpreted in an idealized or excessively formal sense unless clearly defined in the present application.

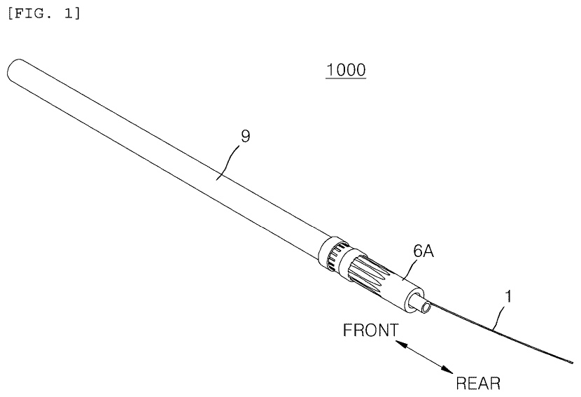

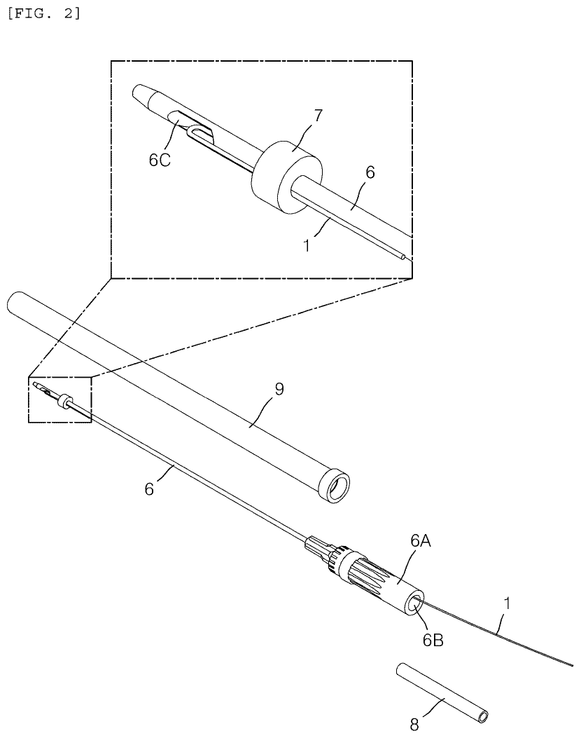

FIG. 1 is a perspective view of a treatment apparatus for lifting skin according to an embodiment of the present disclosure. FIG. 2 is an exploded perspective view of FIG. 1.

With reference to FIGS. 1 and 2, a treatment apparatus for lifting skin 1000 according to an embodiment of the present disclosure may include a thread 1 for a skin lifting treatment, a cannula 6, a first holder 7, a second holder 8, and a cap 9.

The thread 1 for a skin lifting treatment may be inserted through the cannula 6 in the front-to-rear direction. A handle 6A may be provided at a rear end portion of the cannula 6.

A rear end portion of the thread 1 for a skin lifting treatment may be inserted into the cannula 6 through a hole 6C formed on an outer circumferential surface of a front end portion of the cannula 6, and exit rearward from the handle 6A through a hole 6B formed at a rear end of the handle 6A. A front end portion of the thread 1 may be arranged outside the front end portion of the cannula 6 and folded rearward to be overlapped with the outer circumferential surface of the front end portion of the cannula 6. In addition, the front end portion of the thread 1 for a skin lifting treatment may be inserted into the cannula 6 through the hole 6B formed at the rear end of the handle 6A, exit through the hole 6C formed on the outer circumferential surface of the front end portion of the cannula 6, and be folded rearward.

In this state, the front end portion of the cannula 6 may be inserted through the first holder 7 to fix the front end portion of the thread 1. Here, the first holder 7 may be formed of a sponge, but may include any fixing member capable of fixing the front end portion of the thread 1 to the outer circumferential surface of the front end portion of the cannula 6.

After the front end portion of the thread 1 is fixed in this manner, the second holder 8 may be inserted into the hole 6B formed at the rear end of the handle 6A to fix the rear end portion of the thread 1.

Subsequently, the treatment apparatus for lifting skin 1000 may be completed by covering the cannula 6 with the cap 9 and coupling a rear end portion of the cap 9 to a front end portion of the handle 6A, and the treatment apparatus for lifting skin 1000 completed in this manner may be sold as a product.

The cannula 6 functions like a hypodermic needle, and a physician may perform a skin lifting treatment by inserting the cannula 6, with the thread 1 inserted therethrough, into the skin (particularly the face) of a user receiving the skin treatment, fixing the thread 1 within the user's skin, and withdrawing the cannula 6 from the user's skin with a pulling motion to lift the user's skin.

The thread 1 for a skin lifting treatment may be formed of a biodegradable or absorbable polymer material that decomposes and is absorbed into the skin after a certain period in vivo, such as collagen or polymers having an ester-based backbone (or main chain), including PLGA, PLLA, PDO, PLCL, PCL, PHA, PHB, and the like.

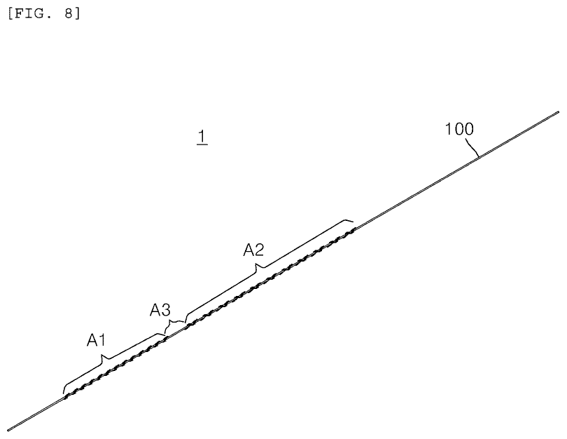

In addition, a plurality of barbs 200 and 300 (see FIG. 9) may protrude from an outer circumferential surface of the thread 1 for a skin lifting treatment. The plurality of barbs 200 and 300 may have a predetermined shape (a three-dimensional shape) capable of enhancing the skin lifting effect for the user.

The thread 1 according to an embodiment of the present disclosure is formed of a bioabsorbable polymer, which develops crystallinity when the predetermined temperature is applied under tension. By utilizing this crystallinity, the thread 1 having the plurality of barbs 200 and 300 with the predetermined shape protruding from the outer circumferential surface may be manufactured.

In particular, the treatment apparatus for lifting skin 1000 according to an embodiment of the present disclosure may be configured to form the thread 1 for a skin lifting treatment within the cannula 6 such that the plurality of barbs 200 and 300 having the predetermined shape protrude from the outer circumferential surface. This allows the apparatus to be sold with the cannula 6, with the thread 1 formed in the cannula 6, without the need to separately manufacture the thread 1 from the cannula 6 for installation into the cannula 6, thereby simplifying the manufacturing process.

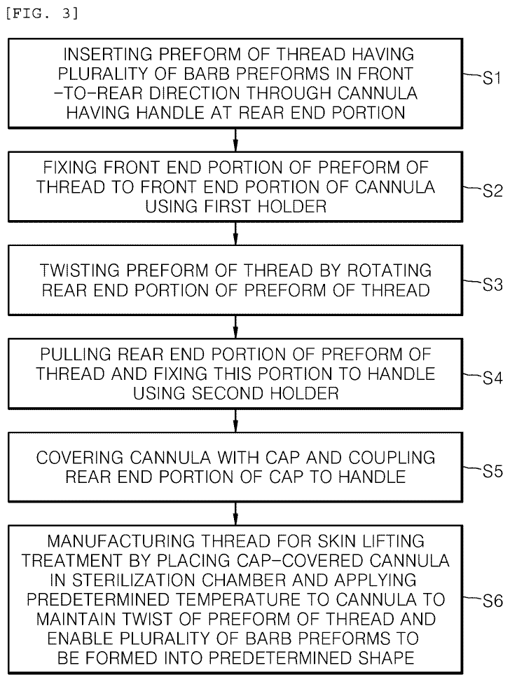

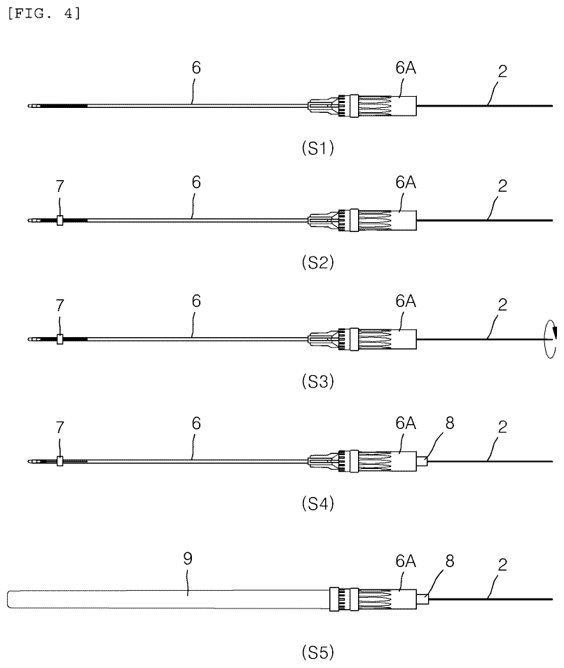

A method of manufacturing the treatment apparatus for lifting skin 1000 according to an embodiment of the present disclosure will be described below with reference to FIGS. 3 and 4.

FIG. 3 is a block diagram illustrating a process according to a method of manufacturing a treatment apparatus for lifting skin according to an embodiment of the present disclosure. FIG. 4 is a view illustrating a process according to a method of manufacturing a treatment apparatus for lifting skin according to an embodiment of the present disclosure.

Referring to FIGS. 3 and 4, the method of manufacturing the treatment apparatus for lifting skin 1000 according to the present disclosure may include steps S1, S2, S3, S4, S5, and S6. Steps S1, S2, S3, S4, S5, and S6 may be performed sequentially.

In step S1, a preform 2 of the thread 1 for a skin lifting treatment may be inserted through the cannula 6, which has the handle 6A at the rear end portion, in the front-to-rear direction. Specifically, in step S1, the rear end portion of the preform 2 of the thread 1 may be inserted into the cannula 6 through the hole 6C formed on the outer circumferential surface of the front end portion of the cannula 6, and exit rearward from the handle 6A through the hole 6B formed at the rear end of the handle 6A, so that the front end portion of the preform 2 of the thread 1 is arranged outside the front end portion of the cannula 6, and the rear end portion of the preform 2 of the thread 1 is arranged outside the handle 6A of the cannula 6. In this manner, the preform 2 of the thread 1 may be inserted through the cannula 6, which has the handle 6A at the rear end portion, in the front-to-rear direction. Alternatively, in step S1, the front end portion of the preform 2 of the thread 1 may be inserted into the cannula 6 through the hole 6B formed at the rear end of the handle 6A and drawn out to the exterior of the front end portion of the cannula 6 through the hole 6C formed on the outer circumferential surface of the front end portion of the cannula 6, so that the front end portion of the preform 2 of the thread 1 is arranged outside the front end portion of the cannula 6, and the rear end portion of the preform 2 of the thread 1 is arranged outside the handle 6A of the cannula 6. In this manner, the preform 2 of the thread 1 may be inserted through the cannula 6, which has the handle 6A at the rear end portion, in the front-to-rear direction.

In step S1, the preform 2 of the thread 1 may be a thread having preforms (not illustrated) of the plurality of barbs 200 and 300 protruding from the outer circumferential surface. Here, the preform 2 of the thread 1 for a skin lifting treatment may refer to a thread temporarily manufactured for the purpose of producing the thread 1 for a skin lifting treatment, and may also refer to the thread 1 for a skin lifting treatment in an initial state. In addition, preforms of the plurality of barbs 200 and 300 refer to the plurality of barbs 200 and 300 in an initial state and may be manufactured by cutting the outer circumferential surface of the preform of the thread 1 for a skin lifting treatment, or may be integrally molded with the preform of the thread 1 for a skin lifting treatment when the preform of the thread 1 for a skin lifting treatment is molded in a mold.

In step S2, the front end portion of the preform 2 of the thread 1 protruding from the front end portion of the cannula 6 may be fixed to the front end portion of the cannula 6 using the first holder 7. Specifically, in step S2, the front end portion of the preform 2 of the thread 1, which passes through the hole 6C (see FIG. 2) formed on the outer circumferential surface of the front end portion of the cannula 6, may be folded rearward, and the front end portion of the cannula 6 may be inserted through the first holder 7 to fix the front end portion of the preform 2 of the thread 1 to the front end portion of the cannula 6 using the first holder 7.

In step S3, the rear end portion of the preform 2 of the thread 1 protruding from the handle 6A may be rotated to twist the preform 2 of the thread 1. Specifically, in step S3, the rear end portion of the preform 2 of the thread 1, which protrudes rearward through the hole 6B (see FIG. 2) formed at the rear end of the handle 6A of the cannula 6, may be rotated to twist the preform 2 of the thread 1.

Twisting the preform 2 of the thread 1 in step S3 serves to evenly arrange the preforms of the plurality of barbs 200 and 300 around the outer circumferential surface of the preform 2 of the thread 1 in the circumferential direction. Specifically, before twisting the preform 2 of the thread 1, when the preform 2 of the thread 1 is viewed from the front or rear, the preforms of the plurality of barbs 200 and 300 may be arranged solely on opposite sides of the outer circumferential surface of the preform 2 of the thread 1 in the circumferential direction. However, after twisting the preform 2 of the thread 1, when the preform 2 of the thread 1 is viewed from the front or rear, the preforms of the plurality of barbs 200 and 300 may be arranged at three or more positions around the outer circumferential surface of the preform 2 of the thread 1 in the circumferential direction.

In step S4, the rear end portion of the preform 2 of the thread 1 may be pulled to tension the preform 2 of the thread 1 and fixed to the handle 6A using the second holder 8. Specifically, in step S4, the rear end portion of the preform 2 of the thread 1, which protrudes rearward through the hole 6B formed at the rear end of the handle 6A of the cannula 6, may be pulled rearward to maintain tension, and the second holder 8 may be inserted into the hole 6B of the handle 6A to fix the rear end portion of the preform 2 of the thread 1 to the handle 6A using the second holder 8. That is, when the second holder 8 is inserted into the hole 6B of the handle 6A, the outer circumferential surface is brought into close contact with the inner circumferential surface of the hole 6B of the handle 6A, thereby fixing the rear end portion of the preform 2 of the thread 1 to the handle 6A.

In step S5, the cap 9 may cover the cannula 6, and the rear end portion of the cap 9 may be coupled to the handle 6A of the cannula 6.

In step S6, a predetermined temperature may be applied to the cannula 6 to maintain the twist of the preform 2 of the thread 1 and to form the preforms of the plurality of barbs 200 and 300 into the plurality of barbs 200 and 300. Here, the predetermined temperature may correspond to a sterilization temperature for the thread 1. That is, the cannula 6, covered with the cap 9 as in step S5, may be placed in a sterilization chamber to sterilize the preform 2 of the thread 1. During this process, the twist of the preform 2 of the thread 1 is maintained, and the preforms of the plurality of barbs 200 and 300 are formed into the three-dimensional shape of the plurality of barbs 200 and 300. Here, when placing the cannula 6 covered with the cap 9 in the sterilization chamber, the cannula 6 covered with the cap 9 may be contained in sterilization packaging and placed in the sterilization chamber.

That is, the thread 1 for a skin lifting treatment is formed of a bioabsorbable polymer, and the polymer develops crystallinity when the predetermined temperature is applied under tension. Accordingly, after pulling the rear end portion of the preform 2 of the thread 1 for a skin lifting treatment rearward and fixing this portion to the handle 6A using the second holder 8, the cannula 6 may be placed in a sterilization chamber. By utilizing the crystallinity formed in the preform 2 of the thread 1 and in the preforms of the plurality of barbs 200 and 300 at the predetermined temperature corresponding to the sterilization temperature, the thread 1 for a skin lifting treatment may be manufactured such that the twist of the preform 2 of the thread 1 is maintained and the preforms of the plurality of barbs 200 and 300 are formed into a predetermined shape.

The thread 1 for a skin lifting treatment, manufactured through steps S1 to S6, may be formed as a twisted thread in which the portion between the first holder 7 and the second holder 8 maintains a twist. The front end portion, relative to the first holder 7, may be formed as a non-twisted thread, and the rear end portion, relative to the second holder 8, may also be formed as a non-twisted thread.

That is, the treatment apparatus for lifting skin 1000 according to an embodiment of the present disclosure may include the cannula 6, which has the handle 6A at the rear end portion, the thread 1 for a skin lifting treatment, which is inserted through the cannula 6 in the front-to-rear direction and has the plurality of barbs 200 and 300 (see FIG. 9) protruding from the outer circumferential surface, the first holder 7 configured to fix the front end portion of the thread 1 protruding from the front end portion of the cannula 6 to the front end portion of the cannula 6, and the second holder 8 configured to fix the rear end portion of the thread 1 protruding from the handle 6A to the handle 6A. In addition, the thread 1 may have the portion between the first holder 7 and the second holder 8 formed as the twisted thread. The front end portion, relative to the first holder 7, may be formed as a non-twisted thread, and the rear end portion, relative to the second holder 8, may also be formed as a non-twisted thread. Hereinafter, the thread 1 for a skin lifting treatment may have the same meaning as the twisted thread, which is the portion between the first holder 7 and the second holder 8 in which the twist is maintained.

In addition, the sterilization chamber may be filled with sterilant gas. That is, the thread 1 may be sterilized using a gas sterilization method. The gas sterilization method is a type of chemical sterilization in which a sterilant is used in a gaseous state or sprayed into the air to sterilize microorganisms. This method is applied to solid materials, equipment, and heat-sensitive disposable plastic medical products. In the present embodiment, the sterilant used is 100% ethylene oxide (EO) gas. In addition to ethylene oxide (EO), other sterilant may be used, such as mixed gases of carbon dioxide with propylene oxide, methyl bromide, formaldehyde, or ozone.

In the present embodiment, the sterilization temperature is 45° C. However, the sterilization temperature may range from 38° C. to 60° C. If the sterilization temperature is below 38° C., the crystallinity may not form. Conversely, if the sterilization temperature exceeds 60° C., the polymer arrangement (crystallinity) may deteriorate, resulting in reduced tensile strength. Accordingly, the sterilization temperature may be in the range of 38° C. to 60° C.

In step S6, sterilization time may range from 90 minutes to 180 minutes. If the sterilization time is shorter than 90 minutes, the crystallinity may form but may not develop completely. Conversely, if the sterilization time exceeds 180 minutes, the crystallinity may not form any further, which may result in unnecessary power consumption in the sterilization chamber. Therefore, the sterilization time may range from 90 minutes to 180 minutes.

The sterilization time of 90 minutes to 180 minutes may refer to actual sterilization time. For example, when the sterilant is the EO gas, time may be required after the sterilization process to purge the toxicity of the EO gas. Since the time required to purge the toxicity of the EO gas alone may be 3 hours to 6 hours, the total time from the start of sterilization to the completion of purging may take 4.5 hours to 9 hours. That is, the sterilization time may range from 1.5 hours (90 minutes) to 3 hours (180 minutes) of actual sterilization time, which is obtained by subtracting the purging time of 3 hours to 6 hours from the total time of 4.5 hours to 9 hours from the start of sterilization to completion of purging.

After completing manufacture of the thread 1 for a skin lifting treatment within the cannula 6 as described above, the cannula 6 covered with the cap 9 may be removed from the sterilization chamber and sold as a product in this state. That is, after completing manufacture of the thread 1 for a skin lifting treatment within the cannula 6 as described above, the sterilization packaging containing the cannula 6 covered with the cap 9 may be removed from the sterilization chamber and sold as a product in this state.

In addition, since the plurality of barbs 200 and 300 of the thread 1 for a skin lifting treatment, manufactured as described above, have a three-dimensional (3D) shape, the skin lifting effect during a skin lifting treatment for a user may be improved compared to a thread for a skin lifting treatment having a plurality of barbs with a conventional two-dimensional (2D) shape.

The present inventor has conducted tests to demonstrate that the thread 1 for a skin lifting treatment having the plurality of 3D-shaped barbs 200 and 300 provides an enhanced skin lifting effect compared to a thread for a skin lifting treatment having a plurality of conventional 2D-shaped barbs. This will be described below with reference to FIGS. 5 to 7.

FIG. 5 is a view illustrating a state in which a thread for a skin lifting treatment of a treatment apparatus for lifting skin according to an embodiment of the present disclosure is inserted into a piece of meat.

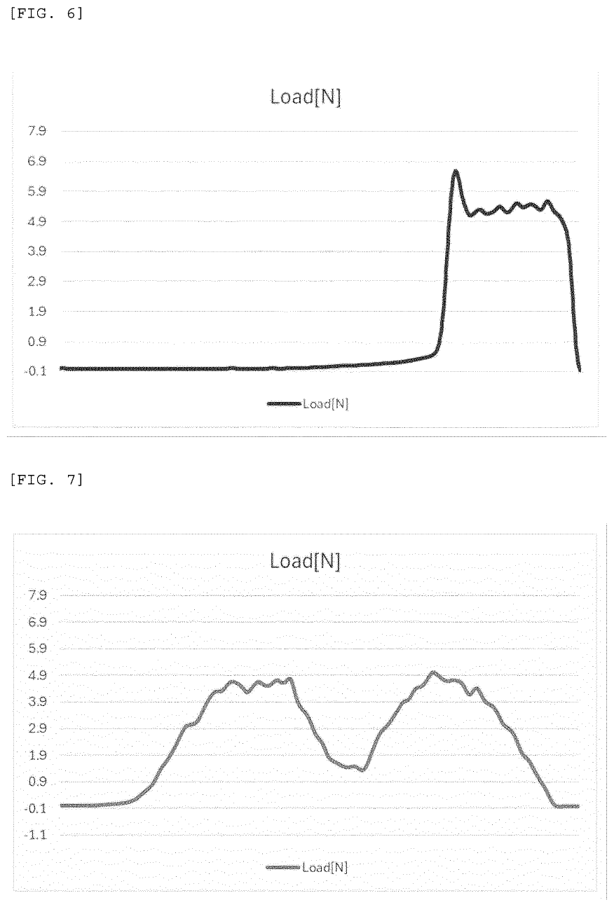

Referring to FIG. 5, the thread 1 for a skin lifting treatment having the plurality of 3D-shaped barbs 200 and 300 is inserted into a piece of meat M. When inserting the thread 1 for a skin lifting treatment into the piece of meat M, a first region A1 of the thread 1 (see FIGS. 8 and 9), which is the actual region for a lifting treatment on the user's sagging skin, is inserted into a piece of meat M.

In addition, a thread for a skin lifting treatment having a plurality of conventional 2D-shaped barbs is inserted into a piece of meat M of the same size and material as the piece of meat M into which the thread 1 for a skin lifting treatment is inserted.

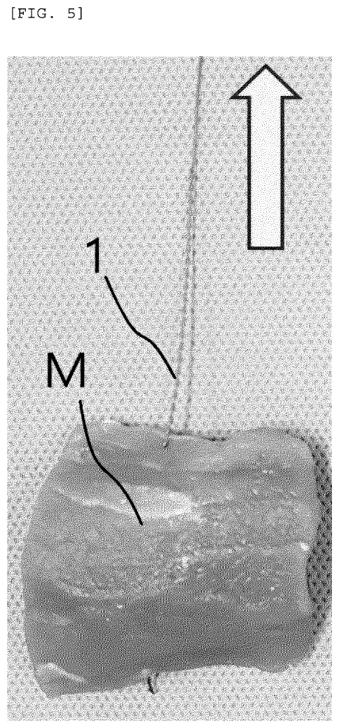

FIG. 6 is a graph illustrating the resistance force measured when a conventional 2D-shaped thread for a skin lifting treatment having a plurality of barbs is inserted in the same manner as in FIG. 5, with the meat piece fixed to a clamp of a tensile tester and the thread pulled in the direction of the arrow. FIG. 7 is a graph illustrating the resistance force measured when a thread for a skin lifting treatment having a plurality of 3D-shaped barbs according to the present disclosure is inserted in the same manner as in FIG. 5, with the meat piece fixed to a clamp of a tensile tester and the thread pulled in the direction of the arrow.

Referring to FIGS. 5 to 7, after fixing the piece of meat M, into which the thread for a skin lifting treatment having the plurality of conventional 2D-shaped barbs is inserted, to a clamp of a tensile tester, the resistance force measured by the tensile tester when the thread for a skin lifting treatment having the plurality of conventional 2D-shaped barbs is pulled in the direction of the arrow is shown in FIG. 6. In addition, after fixing the piece of meat M, into which the thread 1 for a skin lifting treatment having the plurality of 3D-shaped barbs 200 and 300 is inserted, to the clamp of the tensile tester, the resistance force measured by the tensile tester when the thread 1 for a skin lifting treatment having the plurality of 3D-shaped barbs 200 and 300 is pulled in the direction of the arrow is shown in FIG. 7.

That is, the graph of FIG. 6 shows the resistance force measured by the tensile tester reaching a maximum once, and the graph of FIG. 7 shows the resistance force measured by the tensile tester reaching a maximum twice. This indicates that the plurality of conventional 2D-shaped barbs may tear the meat and no longer engage with the meat when the resistance force is measured at a maximum value. However, the plurality of 3D-shaped barbs 200 and 300 engage with the meat once more and the resistance force is measured again even after tearing the meat when the resistance force is first measured at a maximum value.

Accordingly, the thread 1 for a skin lifting treatment having the plurality of 3D-shaped barbs 200 and 300 provides a skin lifting effect compared to the thread for a skin lifting treatment having the plurality of conventional 2D-shaped barbs.

FIG. 8 is a perspective view of the thread for a skin lifting treatment illustrated in FIGS. 1 and 2. FIG. 9 is an enlarged view of a portion of a first, second, and third regions illustrated in FIG. 8.

Referring to FIGS. 8 and 9, the thread 1 for a skin lifting treatment according to an embodiment of the present disclosure may include a thread body 100 and a plurality of barbs 200 and 300 protruding from an outer circumferential surface of the thread body 100.

The thread body 100 may be formed with a predetermined length configured for insertion into a user's skin to perform a skin lifting treatment. The thread 1 for a skin lifting treatment according to an embodiment of the present disclosure has a length ranging from 30 mm to 450 mm. That is, the thread body 100 may have a length ranging from 30 mm to 450 mm. Since faces differ in length, area, and shape among users, the thread body 100 may have various lengths ranging from 30 mm to 450 mm to suit the user's facial contour.

The thread body 100 may have a first region A1 and a second region A2 along the length. Here, the first region A1 may refer to a region in which a plurality of first barbs 200 is protrudingly formed on the outer circumferential surface of the thread body 100, and the second region A2 may refer to a region in which a plurality of first barbs 200 and a plurality of second barbs 300 are protrudingly formed on the outer circumferential surface of the thread body 100.

That is, a plurality of barbs 200 and 300 may include the first barbs 200 and the second barbs 300.

A plurality of first barbs 200 are formed in each of the first region A1 and the second region A2. The first barbs 200 may include a plurality of first barbs 200 formed in the first region A1 and a plurality of first barbs 200 formed in the second region A2.

A plurality of second barbs 300 are formed in the second region A2. The second barbs 300 may include a plurality of second barbs 300 formed in the second region A2.

The first barbs 200 may have end portions directed toward a first direction, and the second barbs 300 may have end portions directed toward a second direction. That is, the end portions of the first barbs 200 may be directed toward the first direction, and the end portions of the second barbs 300 may be directed toward the second direction. Here, the first direction and the second direction may be opposite to each other. That is, the first direction may be opposite to the second direction, and the second direction may be opposite to the first direction.

Specifically, the first direction and the second direction may be a longitudinal direction of the thread body 100. That is, the first direction may be one direction of the longitudinal direction of the thread body 100, and the second direction may be the other direction of the longitudinal direction of the thread body 100. For example, the end portions of the first barbs 200 may be directed toward one direction of the longitudinal direction of the thread body 100, and the end portions of the second barbs 300 may be directed toward the other direction of the longitudinal direction of the thread body 100.

The first region A1 is the actual region for performing a lifting treatment on the user's sagging skin, and the lifting treatment may be performed on the user's sagging skin using the plurality of first barbs 200 arranged in the first region A1. In addition, the second region A2 is a region in which movement forward or rearward along the longitudinal direction of the thread body 100 is restricted, so that the first region A1, after completion of the user's skin lifting treatment, is prevented from moving forward or rearward along the longitudinal direction of the thread body 100. The plurality of second barbs 300 arranged in the second region A2 may prevent the second region A2 from moving toward the first region A1, thereby preventing the first region A1 from sagging again in the direction opposite to the lifting direction. The plurality of first barbs 200 arranged in the second region A2 may prevent the second region A2 from moving in the lifting direction of the first region A1, thereby preventing the plurality of first barbs 200 arranged in the first region A1 from excessively pulling the user's skin after the treatment is completed.

That is, the plurality of first barbs 200 formed in the first region A1 may engage the skin and pull the skin during lifting after the thread 1 is inserted into the skin. The plurality of second barbs 300 formed in the second region A2 may engage the skin after the lifting treatment is completed to prevent the skin from sagging again in the direction opposite to the lifting direction. The plurality of first barbs 200 formed in the second region A2 may prevent only the first barbs 200 in the first region A1 from engaging the skin after the lifting treatment is completed, thereby preventing excessive lifting the skin in the first region A1 alone. Compared to a configuration in which a plurality of first barbs 200 is not formed in the second region A2, the present configuration enables the skin in both the first region A1 and the second region A2 to be lifted during the treatment. As the plurality of first barbs 200 provide overall stimulation, this configuration may enhance collagen production within the skin.

As such, the plurality of first barbs 200 formed in both the first region A1 and the second region A2 have end portions directed toward the first direction, and the plurality of second barbs 300 formed in the second region A2 have end portions directed toward the second direction, which is opposite to the first direction. Accordingly, the first region A1 and the second region A2 at both sides of the longitudinal direction of the thread 1 are supported within the skin, which may improve the lifting effect of the skin and enhance collagen production.

The second region A2 may be longer than the first region A1. However, since faces differ in length, area, and shape among users, the lengths of first region A1 and the second region A2 may be modified as needed. That is, the first region A1 may be longer than the second region A2, or the first region A1 may also have the same length as the second region A2.

In addition, the thread body 100 may further include a third region A3 between the first region A1 and the second region A2 along the length. The outer circumferential surface of the third region A3 may be a region in which a plurality of barbs 200 or 300 is not formed. The third region A3 may be a region that may bend easily during a skin lifting treatment for a user.

If a plurality of first barbs 200 is not protrudingly formed in the second region A2 and only a plurality of second barbs 300 are protrudingly formed, such that the second region A2 has a shape symmetrical to the first region A1, excessive pulling of the lifting thread during a skin lifting treatment for a user may cause the skin in the first region A1 to be lifted up to the second region A2, resulting in a depression in the skin similar to a dimple, or the lifted skin may be engaged with the second region A2 and accumulate in the third region A3. However, in an embodiment of the present disclosure, since the plurality of first barbs 200 and the plurality of second barbs 300 are formed in the second region A2, those problems may be prevented.

Since faces differ in length, area, and shape among users, the third region A3 may have various lengths to suit the user's facial contour. However, in the present embodiment, the third region A3 may be longer than an interval C2 (see FIG. 11) between the plurality of second barb sets S2 (see FIG. 11).

One end portion of the thread body 100 may be arranged closer to the first region A1, and the other end portion of the thread body 100 may be arranged closer to the second region A2. A plurality of barbs 200 and 300 may not be formed on the outer circumferential surface of the one end portion of the thread body 100, and a plurality of barbs 200 and 300 may not be formed on the outer circumferential surface of the other end portion of the thread body 100. The other end portion of the thread body 100 may be longer than the one end portion of the thread body 100. The one end portion of the thread body 100 may be a portion positioned in the direction of insertion into the user's skin during the skin lifting treatment, and the thread body 100 may be inserted into the user's skin from the one end portion.

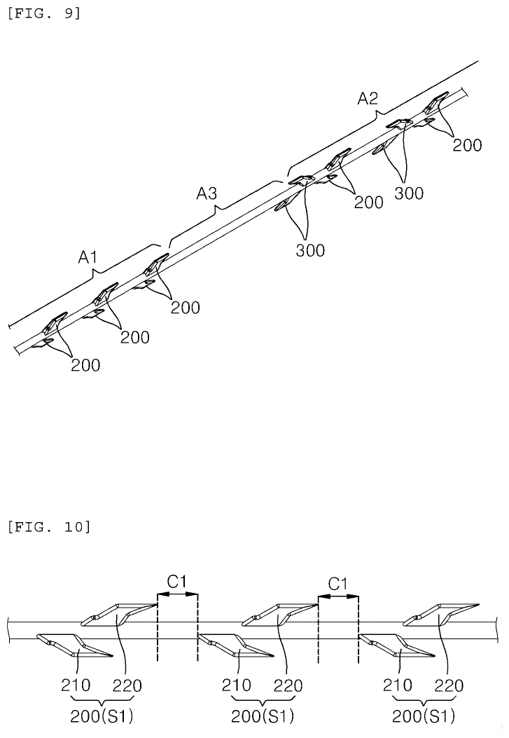

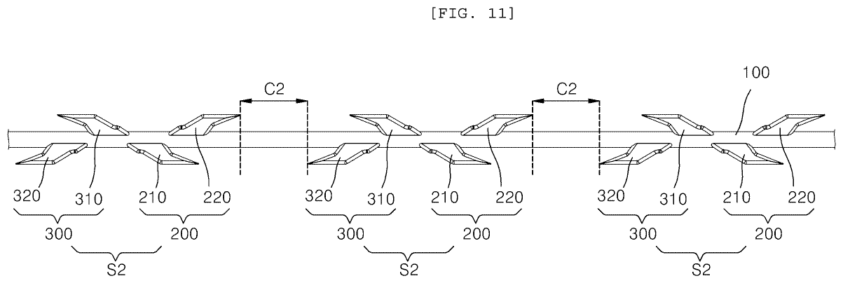

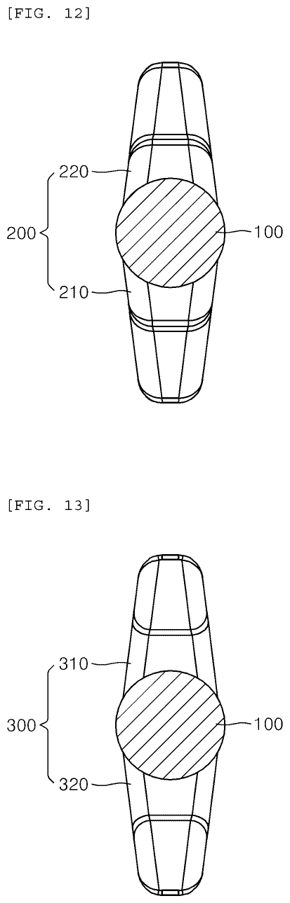

FIG. 10 is an enlarged view of a portion of the first region illustrated in FIG. 8. FIG. 11 is an enlarged view of a portion of the second region illustrated in FIG. 8. FIG. 12 is a left-side view of FIG. 10, illustrating a first barb formed in the first region. FIG. 13 is a left-side view of FIG. 11, illustrating a second barb formed in the second region.

Referring to FIGS. 10 to 13, the first barbs 200 may include a 1-1 barb 210 and a 1-2 barb 220.

The 1-1 barb 210 may be arranged on one side of the outer circumferential surface of the thread body 100, and the 1-2 barb 220 may be arranged on the other side of the outer circumferential surface of the thread body 100.

The 1-2 barb 220 may be arranged adjacent to the 1-1 barb 210 along the longitudinal direction of the thread body 100. That is, the 1-1 barb 210 and the 1-2 barb 220 may be sequentially arranged along the longitudinal direction of the thread body 100, but may be positioned on opposite sides of the outer circumferential surface of the thread body 100.

The 1-1 barb 210 and the 1-2 barb 220 may be sequentially arranged from a distal position in the second region A2 to constitute a first barb set S1. In the first region A1, a plurality of first barb sets S1 may be spaced apart at equal intervals C1 along the length of the thread body 100. That is, a plurality of first barb sets SI may be spaced apart at equal intervals C1 along the length of the thread body 100 in the first region A1.

The second barbs 300 may include a 2-1 barb 310 and a 2-2 barb 320.

The 2-1 barb 310 may be arranged on the opposite side of the outer circumferential surface of the thread body 100, and the 2-2 barb 320 may be arranged on one side of the outer circumferential surface of the thread body 100.

The 2-1 barb 310 may be arranged adjacent to the 1-1 barb 210 along the longitudinal direction of the thread body 100. That is, the 1-1 barb 210 and the 2-1 barb 310 may be sequentially arranged along the longitudinal direction of the thread body 100, but may be positioned on opposite sides of the outer circumferential surface of the thread body 100.

The 2-2 barb 320 may be arranged adjacent to the 2-1 barb 310 along the longitudinal direction of the thread body 100. That is, the 2-1 barb 310 and the 2-2 barb 320 may be sequentially arranged along the longitudinal direction of the thread body 100, but may be positioned on opposite sides of the outer circumferential surface of the thread body 100.

The 2-2 barb 320, the 2-1 barb 310, the 1-1 barb 210, and the 1-2 barb 220 may be sequentially arranged from a position close to the first region A1 to constitute a second barb set S2. In the second region A2, a plurality of second barb sets S2 may be spaced apart at equal intervals C2 along the length of the thread body 100. That is, a plurality of second barb sets S2 may be spaced apart at equal intervals C2 along the length of the thread body 100 in the second region A2.

The interval C2 between the plurality of second barb sets S2 may be greater than the interval C1 between the plurality of first barb sets S1. Since the number of barbs 200 and 300 formed in the second region A2 is greater than the number of barbs 200 formed in the first region A1, the second area A2 may be less likely to bend along the contour of the user's face than the first area A1 during a skin lifting treatment for a user. To address this issue, the interval C2 between the plurality of second barb sets S2 may be greater than the interval C1 between the plurality of first barb sets S1, thereby facilitating bending of the second region A2 during a skin lifting treatment for a user.

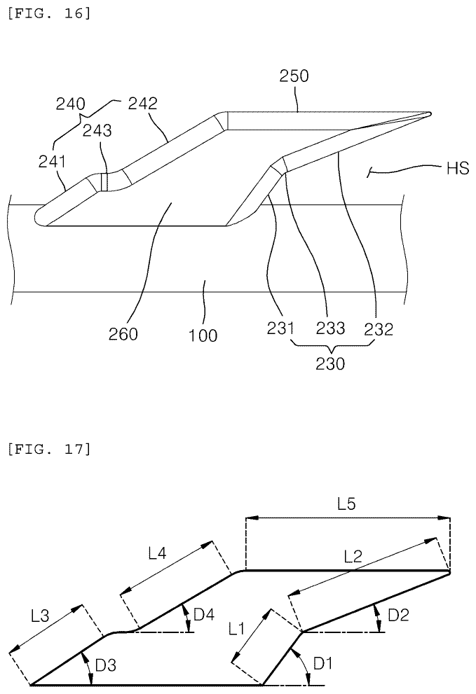

In addition, when a skin lifting treatment for a user is completed using the thread 1, a large amount of the user's internal skin tissue needs to be engaged by the first barbs 200 formed in the first region A1 and by the second barbs 300 formed in the second region A2 in order to enhance the skin lifting effect. That is, the larger a skin-engagement space HS (see FIG. 16) between lifting surfaces 230 of the barbs 200 and 300 (see FIGS. 14 to 16) and the thread body 100, the more the user's skin may engage the skin-engagement space HS during a skin lifting treatment for a user, thereby enhancing the skin lifting effect. In addition, a larger surface area in contact with the internal skin tissue promotes collagen production between the user's skin and the thread when the skin lifting treatment for a user is completed.

The specific structure of the barbs 200 and 300 for enhancing the skin lifting effect will be described below with reference to FIGS. 14 to 17. However, the first barbs 200 and the second barbs 300 may be formed with the same shape and size, with the exception that the end portions thereof extend in opposite directions. That is, the 1-1 barb 210, the 1-2 barb 220, the 2-1 barb 310, and the 2-2 barb 320 may be formed with the same shape and size. Since the first barbs 200 and the second barbs 300 are formed with the same structure in this manner, only the 1-2 barb 220 will be described as an example below and will hereinafter be referred to as the barb 220.

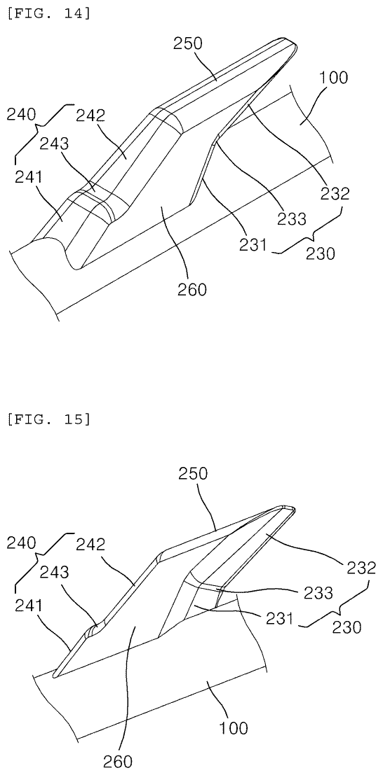

FIG. 14 is a perspective view of a 1-2 barb illustrated in FIGS. 10 and 11. FIG. 15 is a rear-lower perspective view of the 1-2 barb illustrated in FIGS. 10 and 11. FIG. 16 is an enlarged view of the 1-2 barb illustrated in FIGS. 10 and 11.

Referring to FIGS. 14 to 16, the barb 220 may include a concave first bent surface 233 formed on the lifting surface 230. Here, the lifting surface 230 may be a surface on which the user's skin engages during or after a skin lifting treatment for a user. That is, referring to FIG. 9, during a skin lifting treatment for a user, the lifting surfaces 230 of the first barbs 200 formed in the first region A1 engage the user's sagging skin, thereby lifting the sagging skin. After the skin lifting treatment, the lifting surfaces 230 of the second barbs 300 formed in the second region A2 engage the user's skin to support the lifted skin to prevent the skin from sagging again by the lifting surfaces 230 of the first barbs 200 formed in the first region A1.

Since the concave first bent surface 233 is formed on the lifting surface 230, the skin-engagement space HS between the thread body 100 and the lifting surface 230 (see FIG. 16) may be enlarged. This increases the amount of the user's skin that engages the skin-engagement space HS during or after a skin lifting treatment for a user, thereby enhancing the skin lifting effect.

The lifting surface 230 may include a first lifting surface 231 and a second lifting surface 232. The first bent surface 233 may be arranged between the first lifting surface 231 and the second lifting surface 232. The first lifting surface 231 may be arranged closer to the thread body 100 relative to the first bent surface 233. The second lifting surface 232 may be arranged farther from the thread body 100 relative to the first bent surface 233.

The first lifting surface 231 and the second lifting surface 232 may be inclined at different angles relative to the length of the thread body 100. The first lifting surface 231 and the second lifting surface 232 may have a planar configuration. Both lateral portions of the lifting surface 230 may have convex rounded shapes.

The plurality of barbs 220 may have an inclined surface 240 arranged on the side opposite the lifting surface 230, wherein the inclined surface 240 is inclined relative to the length of the thread body 100. During a skin lifting treatment for a user, the physician may perform the treatment by gradually moving the thread 1, positioned within the user's skin, forward and rearward along the longitudinal axis. That is, during a skin lifting treatment for a user, the physician may repeatedly pull the thread 1, positioned within the user's skin, in the lifting direction and push the thread 1 in the opposite direction. The inclined surface 240 is angled to minimize displacement of the user's skin caused by the inclined surface 240 when the physician pushes the thread 1 opposite to the lifting direction.

Specifically, the inclined surface 240 may have a concave second bent surface 243. When the physician pushes the thread 1 opposite to the lifting direction, the second bent surface 243 may prevent the user's skin from being excessively displaced by the inclined surface 240.

The inclined surface 240 may include a first inclined surface 241 and a second inclined surface 242. The second bent surface 243 may be formed on the first inclined surface 241 and the second inclined surface 242. The first inclined surface 241 may be arranged closer to the thread body 100 relative to the second bent surface 243. The second inclined surface 242 may be arranged farther from the thread body 100 relative to the second bent surface 243. The first inclined surface 241 and the second inclined surface 242 may be inclined at different angles relative to the length of the thread body 100. The first inclined surface 241 and the second inclined surface 242 may have a planar configuration. Both lateral portions of the inclined surface 240 may have convex rounded shapes. The area between the first inclined surface 241 and the second bent surface 243 may have a convex rounded shape.

The plurality of barbs 220 may have a parallel surface 250 connecting the terminal edges of the lifting surface 230 and the inclined surface 240, wherein the parallel surface 250 extends parallel to the longitudinal direction of the thread body 100. The parallel surface 250 is arranged parallel to the longitudinal direction of the thread body 100 so that when the physician repeatedly pulls the thread 1 positioned in the skin of the user in the lifting direction and pushes the thread 1 in the opposite direction during a skin lifting treatment for a user, the thread 1 may move easily along the longitudinal direction within the user's skin.

The parallel surface 250 may be the surface of the barb 200 arranged farthest from the thread body 100. The parallel surface 250 may have a planar configuration. Both lateral portions of the parallel surface 250 have convex rounded shapes. The area between the parallel surface 250 and the second lifting surface 232 may have a convex rounded shape. The area between the parallel surface 250 and the second inclined surface 242 may have a convex rounded shape.

Both side surfaces of the plurality of barbs 220 may be formed with planes 260 orthogonal to the parallel surface 250. The plane 260 is formed to be orthogonal to the parallel surface 250, SO that when the physician repeatedly pulls the thread 1 placed in the skin of the user in the lifting direction and pushes the thread 1 in the opposite direction during a skin lifting treatment for a user, the thread 1 may easily move in the longitudinal direction within the skin of the user.

FIG. 17 is a schematic view of the 1-2 barb illustrated in FIG. 16.

Referring to FIGS. 16 and 17, an interior angle D1 of the first lifting surface 231 relative to the length of the thread body 100 may be at least twice an interior angle D2 of the second lifting surface 232 relative to the length of the thread body 100. Through this configuration, during a skin lifting treatment for a user, even if the barb 220 is pushed opposite to the lifting direction by skin engaged in the skin-engagement space HS, the barb 220 may remain fixed without deformation, thereby preventing the skin engaged in the skin-engagement space HS from being released. If the first bent surface 233 is not formed on the lifting surface 230, there is a problem in that the barb 220 needs to be thinner or the barb 220 needs to be increased in size to enlarge the skin-engagement space HS. Therefore, to prevent this problem, the first bent surface 233 may be formed on the lifting surface 230.

In the present embodiment, the interior angle D1 of the first lifting surface 231 relative to the length of the thread body 100 is set to 53.5°, and the interior angle D2 of the second lifting surface 232 relative to the length of the thread body 100 is set to 21.5°. However, the interior angle D1 of the first lifting surface 231 relative to the length of the thread body 100 may range from 25°to 85°, and the interior angle D2 of the second lifting surface 232 relative to the length of the thread body 100 may range from 5° to 60°.

If the interior angle D1 of the first lifting surface 231 relative to the length of the thread body 100 is less than 25°, the skin-engagement space HS may be narrow, which may reduce the skin lifting effect. On the other hand, if the interior angle D1 is greater than 85°, it may be difficult for the thread 1 to move in the longitudinal direction within the user's skin during a skin lifting treatment for a user. Therefore, the interior angle Dl of the first lifting surface 231 relative to the length of the thread body 100 may range from 25°to 85°.

If the interior angle D2 of the second lifting surface 232 relative to the length of the thread body 100 is less than 5°, the skin-engagement space HS may be narrow, which may reduce the skin lifting effect. On the other hand, if the interior angle D2 is greater than 60°, it may be difficult for the thread 1 to move in the longitudinal direction within the user's skin during a skin lifting treatment for a user. Therefore, the interior angle D2 of the second lifting surface 232 relative to the length of the thread body 100 may range from 5°to 60°.

The interior angle D3 of the first inclined surface 241 relative to the length of the thread body 100 may be greater than an interior angle D4 of the second inclined surface 242 relative to the length of the thread body 100. Through this configuration, among the widths of the barb 220 in the longitudinal direction of the thread body 100, the width of the portion closer to the thread body 100 is formed wider than the width of the portion farther from the thread body 100, thereby preventing the barb 220 from easily deforming during a skin lifting treatment for a user.

In the present embodiment, the interior angle D3 of the first inclined surface 241 relative to the length of the thread body 100 is set to 33.6°, and the interior angle D4 of the second inclined surface 242 relative to the length of the thread body 100 is set to 30.3°. However, the interior angle D3 of the first inclined surface 241 relative to the length of the thread body 100 may range from 25°to 85°, and the interior angle D4 of the second inclined surface 242 relative to the length of the thread body 100 may range from 5° to 60°.

If the interior angle D3 of the first inclined surface 241 relative to the length of the thread body 100 is less than 25°, the width of the barb 220 in the longitudinal direction of the thread body 100 may be narrow, which may cause the barb 220 to deform easily during a skin lifting treatment for a user. On the other hand, if the interior angle D3 is greater than 85°, it may be difficult for the thread 1 to move in the longitudinal direction within the user's skin during a skin lifting treatment for a user. Therefore, the interior angle D3 of the first inclined surface 241 relative to the length of the thread body 100 may range from 25°to 85°.

If the interior angle D4 of the first inclined surface 242 relative to the length of the thread body 100 is less than 5°, the width of the barb 220 in the longitudinal direction of the thread body 100 may be narrow, which may cause the barb 220 to deform easily during a skin lifting treatment for a user. On the other hand, if the interior angle D4 is greater than 60°, it may be difficult for the thread 1 to move in the longitudinal direction within the user's skin during a skin lifting treatment for a user. Therefore, the interior angle D4 of the second inclined surface 242 relative to the length of the thread body 100 may range from 5°to 60°.

The interior angle D1 of the first lifting surface 231 relative to the length of the thread body 100 may be greater than the interior angle D3 of the first inclined surface 241 relative to the length of the thread body 100, and the interior angle D4 of the second inclined surface 242 relative to the length of the thread body 100 may be greater than the interior angle D2 of the second lifting surface 232 relative to the length of the thread body 100. In other words, the interior angle D2 of the second lifting surface 232 relative to the length of the thread body 100, the interior angle D4 of the second inclined surface 242 relative to the length of the thread body 100, the interior angle D3 of the first inclined surface 241 relative to the length of the thread body 100, and the interior angle D1 of the first lifting surface 231 relative to the length of the thread body 100 may increase sequentially.

A protrusion length L2 of the second lifting surface 232 may be longer than a protrusion length L1 of the first lifting surface 231. Through this configuration, the skin-engagement space HS may be enlarged, thereby enhancing the skin lifting effect during a skin lifting treatment for a user.

In the present embodiment, the protrusion length L1 of the first lifting surface 231 is set to 0.1395 mm, and the protrusion length L2 of the second lifting surface 232 is set to 0.5770 mm. However, the protrusion length L1 of the first lifting surface 231 may range from 0.0395 mm to 0.2395 mm, and the protrusion length L2 of the second lifting surface 232 may range from 0.4770 mm to 0.6770 mm.

If the protrusion length L1 of the first lifting surface 231 is shorter than 0.0395 mm, the skin-engagement space HS may be narrow, which may reduce the skin lifting effect. On the other hand, if the protrusion length L1 is longer than 0.2395 mm, the barb 220 may deform easily during a skin lifting treatment for a user. Therefore, the protrusion length L1 of the first lifting surface 231 may range from 0.0395 mm to 0.2395 mm.

If the protrusion length L2 of the second lifting surface 232 is shorter than 0.4770 mm, the skin-engagement space HS may be narrow, which may reduce the skin lifting effect. On the other hand, if the protrusion length L2 is longer than 0.6770 mm, the barb 220 may deform easily during a skin lifting treatment for a user. Therefore, the protrusion length L2 of the second lifting surface 232 may range from 0.4770 mm to 0.6770 mm.

A protrusion length L4 of the second inclined surface 242 may be longer than a protrusion length L3 of the first inclined surface 241. Through this configuration, the inclined surface 240 has a low inclination angle, allowing the thread 1 to move easily in the longitudinal direction within the user's skin during a skin lifting treatment for a user.

In the present embodiment, the protrusion length L3 of the first inclined surface 241 is set to 0.2004 mm, and the protrusion length L4 of the second inclined surface 242 is set to 0.4315 mm. However, the protrusion length L3 of the first inclined surface 241 may range from 0.1004 mm to 0.3004 mm, and the protrusion length L4 of the second inclined surface 242 may range from 0.3315 mm to 0.5315 mm.

If the protrusion length L3 of the first inclined surface 241 is shorter than 0.1004 mm, the inclined surface 240 may have a high inclination angle, which may cause the user's skin to be pushed by the inclined surface 240 during a skin lifting treatment for a user. On the other hand, if the protrusion length L3 is longer than 0.3004 mm, the barb 220 may deform easily during a skin lifting treatment for a user. Therefore, the protrusion length L3 of the first inclined surface 241 may range from 0.1004 mm to 0.3004 mm.

If the protrusion length L4 of the second inclined surface 242 is shorter than 0.3315 mm, the inclined surface £ 240 may have a high inclination angle, which may cause the user's skin to be pushed by the inclined surface 240 during a skin lifting treatment for a user. On the other hand, if the protrusion length L4 is longer than 0.5315 mm, the barb 220 may deform easily during a skin lifting treatment for a user. Therefore, the protrusion length L4 of the second inclined surface 242 may range from 0.3315 mm to 0.5315 mm.

A length L5 of the parallel surface 250 in the longitudinal direction of the thread body 100 may longer than the protrusion length L2 of the second lifting surface 232.

In the present embodiment, the length L5 of the parallel surface 250 in the longitudinal direction of the thread body 100 is set to 0.7431 mm. However, the length L5 of the parallel surface 250 in the longitudinal direction of the thread body 100 may range from 0.6431 mm to 0.8431 mm.

If the length L5 of the parallel surface 250 in the longitudinal direction of the thread body 100 is shorter than 0.6431 mm, the width of the barb 220 in the longitudinal direction of the thread body 100 may be narrow, which may cause the barb 220 to deform easily during a skin lifting treatment for a user. On the other hand, if the length L5 is longer than 0.8431 mm, the width of the barb 220 in the longitudinal direction of the thread body 100 may be wide, which may cause the user's skin to be pushed by the inclined surface 240 during a skin lifting treatment for a user. Therefore, the length L5 of the parallel surface 250 in the longitudinal direction of the thread body 100 may range from 0.6431 mm to 0.8431 mm.

The protrusion length L3 of the first inclined surface 241 may be longer than the protrusion length L1 of the first lifting surface 231, the protrusion length L2 of the second lifting surface 232 may be longer than the protrusion length L4 of the second inclined surface 242, and the length L5 of the parallel surface 250 in the longitudinal direction of the thread body 100 may be longer than the protrusion length L2 of the second lifting surface 232. In other words, the protrusion length L1 of the first lifting surface 231, the protrusion length L3 of the first inclined surface 241, the protrusion length L4 of the second inclined surface 242, the protrusion length L2 of the second lifting surface 232, and the length L5 of the parallel surface 250 in the longitudinal direction of the thread body 100 may increase sequentially.

As described above, with the manufacturing method of the treatment apparatus for lifting skin 1000 according to the embodiment of present disclosure and the treatment apparatus for lifting skin 1000 manufactured using the same, the thread 1 for a skin lifting treatment, having a plurality of barbs 200 and 300, may be formed within the cannula 6 for commercial sale. This allows the thread 1 to be sold with the cannula 6 in a configuration formed within the cannula 6, without the need to separately manufacture the thread 1 from the cannula 6 and install the thread in the cannula 6, thereby simplifying the manufacturing process.

Those skilled in the art to which the present disclosure pertains will understand that the present disclosure may be implemented in other specific forms without changing the technical spirit or essential characteristics of the present disclosure. Accordingly, the embodiments described above are to be understood as illustrative in all respects and not limiting. The scope of the present disclosure is defined by the appended claims rather than by the detailed description, and all changes or modifications derived from the meanings and scope of the claims and equivalent concepts thereof should be construed as being included in the scope of the present disclosure.

### Industrial Applicability

The present disclosure provides a method of manufacturing a treatment apparatus for lifting skin in which a thread for a skin lifting treatment having a plurality of barbs may be formed within a cannula, and a treatment apparatus for lifting skin manufactured using the same.

1. A method of manufacturing a treatment apparatus for lifting skin, the method comprising:
(S1) inserting a preform of a thread for a skin lifting treatment, wherein a plurality of barb preforms is protrudingly formed on an outer circumferential surface, in a front-to-rear direction through a cannula having a handle at a rear end portion;
(S2) fixing a front end portion of the preform of the thread protruding from a front end portion of the cannula to the front end portion of the cannula using a first holder;
(S3) twisting the preform of the thread by rotating a rear end portion of the preform of the thread protruding from the handle;
(S4) pulling the rear end portion of the preform of the thread to tension the preform of the thread and fixing the rear end portion of the preform of the thread to the handle using a second holder;
(S5) covering the cannula with a cap and coupling the rear end portion of the cap to the handle; and
(S6) applying a predetermined temperature to the cannula to maintain a twist of the preform of the thread and to form the plurality of barb preforms into the plurality of barbs.

2. The method of claim 1, wherein the predetermined temperature is a sterilization temperature of the thread.

3. The method of claim 2, wherein the sterilization temperature ranges from 38° C. to 60° C.

4. The method of claim 3, wherein in S6, sterilization time ranges from 90 minutes to 180 minutes.

5. The method of claim 2, wherein in S6, the cannula, covered with the cap and contained in sterilization packaging, is placed in a sterilization chamber and the predetermined temperature is applied.

6. The method of claim 1, wherein the plurality of formed barbs in S6 includes a lifting surface configured to engage a user's internal skin tissue between the barbs and the thread body,
wherein the lifting surface includes a concave first bent surface.

7. A treatment apparatus for lifting skin manufactured using the method according to claim 1.

8. A treatment apparatus for lifting skin, the treatment apparatus, comprising:
a cannula having a handle at a rear end portion;
a thread for a skin lifting treatment, having a plurality of barbs protrudingly formed on an outer circumferential surface and configured to be inserted in a front-to-rear direction through the cannula;
a first holder configured to fix a front end portion of the thread protruding from a front end portion of the cannula to the front end portion of the cannula; and
a second holder configured to fix a rear end portion of the tread protruding from the handle to the handle,
wherein the thread is formed such that a portion between the first holder and the second holder is formed as twisted thread, and the front end portion relative to the first holder and the rear end portion relative to the second holder are formed as untwisted thread.

9. The treatment apparatus of claim 8, wherein the plurality of barbs comprises:
a lifting surface;
an inclined surface arranged on a side opposite the lifting surface and inclined relative to a length of the thread body;
a parallel surface connecting terminal edges of the lifting surface and the inclined surface, parallel to the length of the thread body; and
both side surfaces having planar configurations.

10. The treatment apparatus of claim 9, wherein a concave first bent surface is formed on the lifting surface of the plurality of barbs.

11. The treatment apparatus of claim 10, wherein the lifting surface comprises:
a first lifting surface arranged closer to the thread body relative to the first bent surface; and
a second lifting surface arranged farther from the thread body relative to the first bent surface,
wherein the first lifting surface and the second lifting surface are arranged and inclined at different angles relative to the length of the thread body.

12. The treatment apparatus of claim 11, wherein an interior angle of the first lifting surface relative to the length of the thread body is at least twice an interior angle of the second lifting surface relative to the length of the thread body.

13. The treatment apparatus of claim 12, wherein a protrusion length of the second lifting surface is longer than a protrusion length of the first lifting surface.

14. The treatment apparatus of claim 9, wherein respective lateral portions of the lifting surface, the inclined surface, and the parallel surface have a convex rounded shape.

15. The treatment apparatus of claim 9, wherein the inclined surface includes a concave second bent surface.

16. The treatment apparatus of claim 15, wherein the inclined surface comprises:
a first inclined surface arranged closer to the thread body relative to the second bent surface; and
a second inclined surface arranged farther from the thread body relative to the second bent surface,
wherein the first inclined surface and the second inclined surface are arranged and inclined at different angles relative to the length of the thread body.

17. The treatment apparatus of claim 16, wherein an interior angle of the first inclined surface relative to the length of the thread body is greater than an interior angle of the second inclined surface relative to the length of the thread body.

18. The treatment apparatus of claim 17, wherein a protrusion length of the second inclined surface is longer than a protrusion length of the first inclined surface.
